(19) United States
(12) Patent Application Publication
(10) Pub. No.: US 2016/0115101 A1
(43) Pub. Date: Apr. 28, 2016

(54) METHODS AND APPARATUSES FOR REFORMING OF HYDROCARBONS INCLUDING RECOVERY OF PRODUCTS USING MIXING DEVICES

(71) Applicant: UOP LLC, Des Plaines, IL (US)

(72) Inventors: William Yanez, Crystal Lake, IL (US); Lisa Lane, Elk Grove, IL (US); David William Ablin, Arlington Heights, IL (US); Robert Edison Tsai, Arlington Heights, IL (US); Xin X. Zhu, Long Grove, IL (US); Tokhanh Ngo, Glendale Heights, IL (US)

(21) Appl. No.: 14/524,271

(22) Filed: Oct. 27, 2014

(51) Int. Cl. C07C 7/04; C10L 3/12; B01D 53/14; B01D 3/14; B01D 3/26; C07C 7/11; C01B 3/50

(52) U.S. Cl. CPC C07C 7/04; C07C 7/11; C10L 3/12; C01B 3/508; B01D 3/143; B01D 3/26; B01D 53/1487; C10L 2290/543; C10L 2290/542; C10L 2290/24; C10L 2290/30; C01B 2203/048; C01B 2203/0415; B01D 2252/504

(57) ABSTRACT

Embodiments of apparatuses and methods for reforming of hydrocarbons including recovery of products are provided. In one example, an apparatus comprises a separation zone to receive and separate a reforming-zone effluent to form a net gas phase stream and a liquid phase hydrocarbon stream. A compressor receives and compresses the net gas phase stream to form a compressed net gas phase stream. A chiller receives and cools the liquid phase hydrocarbon stream to form a cooled liquid phase hydrocarbon stream. A first mixing device receives and mixes the compressed net gas phase stream and at least a portion of the cooled liquid phase hydrocarbon stream to extract C₃/C₄ hydrocarbons from the compressed net gas phase stream into the at least the portion of the cooled liquid phase hydrocarbon stream.

### TECHNICAL FIELD

The technical field relates generally to reforming of hydrocarbons, and more particularly relates to apparatuses and methods for reforming of hydrocarbons with improved recovery of products from a reforming-zone effluent using mixing devices.

### BACKGROUND

High octane gasoline is needed for modern gasoline engines. Previously, octane numbers were often improved by incorporating various lead-containing additives into the gasoline. As lead-containing additives have been phased out of gasoline for environmental reasons, it has become increasingly necessary to rearrange the structure of the hydrocarbons used in gasoline blending to achieve higher octane ratings. Catalytic reforming of hydrocarbons is a process widely used by refiners for upgrading the octane ratings of gasoline as well as for other useful hydrocarbon conversion applications.

In catalytic reforming, a hydrocarbon feedstock of, for example, C₅ hydrocarbons to about C₁₁ hydrocarbons, is contacted with a reforming catalyst to convert at least a portion of the heavier hydrocarbons to aromatic hydrocarbons, for example, to increase the octane content of gasoline. The catalytic reforming of the heavier hydrocarbons to produce a reformate that includes aromatic hydrocarbons also produces significant quantities of valuable hydrogen and lighter hydrocarbons, such as liquefied petroleum gas (LPG) containing primarily C₃ and C₄ hydrocarbons. Refiners are looking for ways to maximize the recovery of reforming products, such as reformate, hydrogen and LPG, from the reforming reactor effluent.

Accordingly, it is desirable to provide apparatuses and methods for reforming of hydrocarbons with improved recovery of products from a reforming reactor effluent. Furthermore, other desirable features and characteristics of the present invention will become apparent from the subsequent detailed description and the appended claims, taken in conjunction with the accompanying drawings and this background.

### BRIEF SUMMARY

Apparatuses and methods for reforming of hydrocarbons including recovery of products are provided herein. In accordance with an exemplary embodiment, an apparatus for reforming of hydrocarbons including recovery of products comprises a separation zone that is configured to receive and separate a reforming-zone effluent that comprises H₂, C₄⁻ hydrocarbons, and C₅⁺ hydrocarbons including aromatics to form a net gas phase stream and a liquid phase hydrocarbon stream. The net gas phase stream comprises H₂ and C₆⁻ hydrocarbons and the liquid phase hydrocarbon stream comprises C₅⁺ hydrocarbons. A first compressor is configured to receive and compress the net gas phase stream to form a compressed net gas phase stream. A chiller is configured to receive and cool the liquid phase hydrocarbon stream to form a cooled liquid phase hydrocarbon stream. A first mixing device is configured to receive and mix the compressed net gas phase stream and at least a portion of the cooled liquid phase hydrocarbon stream to extract C₃/C₄ hydrocarbons from the compressed net gas phase stream into the at least the portion of the cooled liquid phase hydrocarbon stream to form a first two-phase combined stream.

In accordance with another exemplary embodiment, a method for reforming of hydrocarbons including recovery of products is provided. The method comprises the steps of separating a reforming-zone effluent that comprises H₂, C₄⁻ hydrocarbons, and C₅⁺ hydrocarbons including aromatics to form a net gas phase stream and a liquid phase hydrocarbon stream. The net gas stream comprises H₂ and C₆⁻ hydrocarbons and the liquid phase hydrocarbon stream comprises C₅⁺ hydrocarbons. The net gas phase stream is compressed to form a compressed net gas phase stream. The liquid phase hydrocarbon stream is cooled to form a cooled liquid phase hydrocarbon stream. The compressed net gas phase stream is mixed with at least a portion of the cooled liquid phase hydrocarbon stream in a mixing device to extract C₃/C₄ hydrocarbons from the compressed net gas phase stream into the at least the portion of the cooled liquid phase hydrocarbon stream to form a first two-phase combined stream.

In accordance with another exemplary embodiment, a method for reforming of hydrocarbons including recovery of products is provided. The method comprises the steps of mixing a gas phase stream that comprises H₂ and C₆⁻ hydrocarbons with a liquid phase hydrocarbon stream that comprises C₅⁺ hydrocarbons in a jet mixer to extract C₃/C₄ hydrocarbons from the gas phase stream into the liquid phase hydrocarbon stream and to form a first two-phase combined stream. The first two-phase combined stream is separated for forming an H₂-rich stream that comprises primarily H₂, a C₃/C₄ hydrocarbon-rich LPG stream that comprises primarily C₃/C₄ hydrocarbons, and a C₅⁺ hydrocarbon-rich reformate stream that comprises primarily C₅⁺ hydrocarbons.

### BRIEF DESCRIPTION OF THE DRAWINGS

The various embodiments will hereinafter be described in conjunction with the following drawing figures, wherein like numerals denote like elements, and wherein:

FIG. 1 schematically illustrates an apparatus and a method for reforming of hydrocarbons including recovery of products in accordance with an exemplary embodiment; and

FIG. 2 schematically illustrates an apparatus and a method for reforming of hydrocarbons including recovery of products in accordance with another exemplary embodiment.

### DETAILED DESCRIPTION

The following Detailed Description is merely exemplary in nature and is not intended to limit the various embodiments or the application and uses thereof. Furthermore, there is no intention to be bound by any theory presented in the preceding background or the following detailed description.

Various embodiments contemplated herein relate to apparatuses and methods for reforming of hydrocarbons with improved recovery of products from a reforming-zone effluent. The exemplary embodiments taught herein provide a separation zone in fluid communication with a reforming zone to receive a reforming-zone effluent. As used herein, the term “zone” refers to an area including one or more equipment items and/or one or more sub-zones. Equipment items can include one or more reactors or reactor vessels, scrubbers, strippers, fractionators or distillation columns, absorbers or absorber vessels, regenerators, heaters, exchangers, coolers/chillers, pipes, pumps, compressors, controllers, and the like. Additionally, an equipment item can further include one or more zones or sub-zones. The reforming-zone effluent comprises hydrogen (H₂), C₄⁻ hydrocarbons, and C₅⁺ hydrocarbons including aromatics. As used herein, Cₓ means hydrocarbon molecules that have “X” number of carbon atoms, Cₓ⁺ means hydrocarbon molecules that have “X” and/or more than “X” number of carbon atoms, and Cₓ⁻ means hydrocarbon molecules that have “X” and/or less than “X” number of carbon atoms.

The separation zone separates the reforming-zone effluent to form a net gas phase stream and a liquid phase hydrocarbon stream. The net gas phase stream comprises H₂ and C₆⁻ hydrocarbons and the liquid phase hydrocarbon stream comprises C₅⁺ hydrocarbons. In an exemplary embodiment, a compressor compresses the net gas phase stream to form a compressed net gas phase stream and a chiller cools the liquid phase hydrocarbon stream to form a cooled liquid phase hydrocarbon stream.

A mixing device, such as a jet mixer or the like, is used to mix the compressed net gas phase stream and at least a portion of the cooled liquid phase hydrocarbon stream to form a two-phase combined stream. In an exemplary embodiment, the mixing device aggressively mixes or homogenizes the net gas phase stream into the cooled liquid phase hydrocarbon stream to deliver the two-phase combined stream in a turbulent flow regime (e.g., nearly ideal mixing conditions) as opposed to a transitional or laminar flow regime that might otherwise result in stratified flow (e.g., layered laminar flow or relatively poor mixing conditions). Additionally, in an exemplary embodiment, the liquid phase hydrocarbon stream is rich in C₅⁺ hydrocarbons that readily extract C₃/C₄ hydrocarbons from the net gas phase stream. As such, by aggressively mixing the compressed net gas phase stream and at least a portion of the cooled liquid phase hydrocarbon stream, C₃/C₄ hydrocarbons are effectively extracted from the compressed net gas phase to the cooled liquid phase with C₅⁺ hydrocarbons to form the two-phase combined stream having a gas phase that is rich in H₂ and substantially depleted of C₃⁺ hydrocarbons and a liquid phase that is rich in C₃⁺ hydrocarbons. In an exemplary embodiment, the two-phase combined stream is separated by further separating the gas and liquid phases to form an H₂-rich stream that comprises primarily H₂, a C₃/C₄ hydrocarbon-rich LPG stream that comprises primarily C₃/C₄ hydrocarbons, and a C₅⁺ hydrocarbon-rich reformate stream that comprises primarily C₅⁺ hydrocarbons.

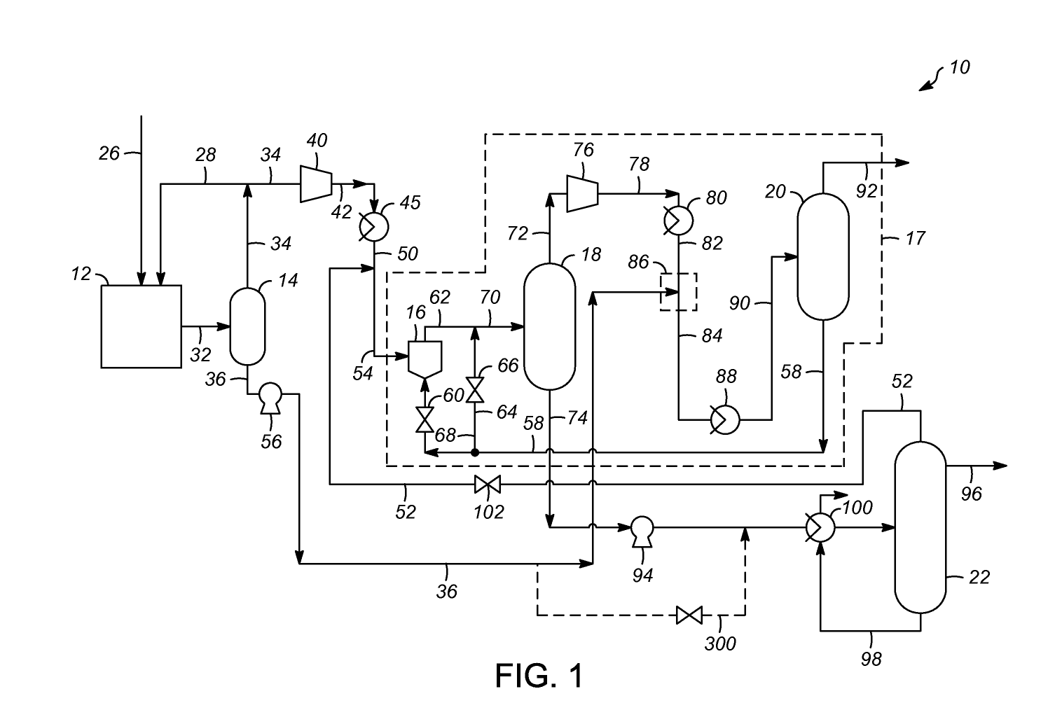

Referring to FIG. 1, an apparatus 10 for reforming of hydrocarbons in accordance with an exemplary embodiment is provided. The apparatus 10 comprises a reforming zone 12, a separation zone 14, a recontacting zone 17 including a mixing device 16 and recontact drums 18 and 20, and a stabilizer 22 that are in fluid communication.

In an exemplary embodiment, a reforming-zone feedstock 26 containing naphtha fraction hydrocarbons, such as from C₅ to about C₁₁ hydrocarbons with a boiling point range of, for example, from about 70 to about 205° C., is introduced to the apparatus 10. The reforming-zone feedstock 26 and a recycle net gas phase stream 28 (discussed in further detail below) are passed along to the reforming zone 12 that contains a reforming catalyst as is well-known in the art. The reforming zone 12 will typically comprise a plurality of stacked or side-by-side reactors with provisions for intermediate heating of the intermediate reactant stream (e.g., the reforming-zone feedstock 26 and the recycle net gas phase stream 28 including any conversion products formed therefrom) and one or more heat exchangers. In an exemplary embodiment, in the reforming zone 12, the recycle net gas phase stream 28 is combined with the reforming-zone feedstock 26 for contact with the reforming catalyst.

A reforming-zone effluent 32 is formed in the reforming zone 12 and contains H₂, C₅⁺ hydrocarbons including aromatics, and lighter hydrocarbons such as C₄⁻ hydrocarbons including C₃ and C₄ hydrocarbons. In an exemplary embodiment, the reforming-zone effluent 32 is a two-phase liquid-gas stream that is relatively hot in which H₂ and the lighter hydrocarbons (e.g., C₄⁻ hydrocarbons) are predominately in the gas phase and the heavier hydrocarbons (e.g., C₅⁺ hydrocarbons including aromatics) are predominately in the liquid phase. In one embodiment, the reforming-zone effluent 32 has a temperature of at least about 35° C., such as from about 35 to about 50° C.

The reforming-zone effluent 32 is introduced to the separation zone 14. The separation zone 14 separates the reforming-zone effluent 32 into net gas phase stream 34 and a liquid phase hydrocarbon stream 36. In an exemplary embodiment, the net gas phase stream 34 comprises H₂ and C₆⁻ hydrocarbons and the liquid phase hydrocarbon stream 36 comprises C₅⁺ hydrocarbons including aromatics. In one example, the net gas phase stream 34 comprises H₂ present in an amount of from about 80 to about 90 mole %, C₁ hydrocarbons present in an amount of about 2 to about 5 mole %, C₂ hydrocarbons present in an amount of from about 2 to about 5 mole %, C₃ hydrocarbons present in an amount of from about 2 to about 4 mole %, C₄ hydrocarbons present in an amount of from about 1.5 to about 2.5 mole %, and possibly some C₅⁺ hydrocarbons. In another example, the liquid phase hydrocarbon stream 36 comprises C₅⁺ hydrocarbons present in an amount of from about 90 to about 99.9 mole % and possibly some C₄⁻ hydrocarbons and H₂. In an exemplary embodiment, the separation zone 14 is operated at a temperature of from about 35 to about 50° C. and a pressure of from about 240 to about 830 kPa gauge.

A portion of the net gas phase stream 34 is passed back to the reforming zone 12 as the recycle net gas phase stream 28 as discussed above and a remaining portion of the net gas phase stream 34 is passed along to a compressor 40. The compressor 40 compresses the net gas phase stream 34 to form a compressed net gas phase stream 42. In an exemplary embodiment, the compressed net gas phase stream 42 has a temperature of from about 120 to about 150° C. and, independently, a pressure of from about 720 to about 2,490 kPa gauge.

The compressed net gas phase stream 42 is passed along to a cooler 45. In the cooler 45, the compressed net gas phase stream 42 is partially cooled to form a partially cooled, compressed net gas phase stream 50. In an exemplary embodiment, the partially cooled, compressed net gas phase stream 50 has a temperature of from about 30 to about 65° C. and, independently, a pressure of from about 690 to about 2,460 kPa gauge.

A light ends stabilizer stream 52 comprising H₂ and C₂⁻ hydrocarbons is passed along from the stabilizer 22 as will be discussed in further detail below and is combined with the partially cooled, compressed net gas phase stream 50 to form a combined stream 54. In an exemplary embodiment, the combined stream has a temperature of from about 30 to about 65° C. and, independently, a pressure of from about 690 to about 2,460 kPa gauge. The combined stream 54 is passed along to the recontacting zone 17 and introduced to the mixing device 16.

The liquid phase hydrocarbon stream 36 exits the separation zone 14 and is passed through a pump 56 and introduced to the recontacting zone 17. The recontacting zone 17 may be configured as a countercurrent gas and liquid phase recontacting zone for further separating H₂, C₃/C₄ hydrocarbons, and/or C₅⁺ hydrocarbons via extraction and/or absorption by contacting the liquid and gas phase fractions of the combined stream 54 and the liquid phase hydrocarbon stream 36. Alternatively, the recontacting zone 17 is not limited to countercurrent flow and that other modes, such as co-current modes as are known in the art, may be used for the recontacting zone 17.

In an exemplary embodiment, a cooled intermediate liquid phase hydrocarbon stream 58 is formed, such as may be produced from a gas-liquid separation, in the recontacting zone 17. The cooled intermediate liquid phase hydrocarbon stream 58 comprises C₃/C₄ hydrocarbons and is rich in C₅⁺ hydrocarbons. In an exemplary embodiment, the cooled intermediate liquid phase hydrocarbon stream 58 has a temperature of from about −28 to about 4° C., such as about −12 to about 0° C., and, independently, a pressure of from about 1,920 to about 5,520 kPa gauge.

The cooled intermediate liquid phase hydrocarbon stream 58 is passed through a valve 60 and is introduced to the mixing device 16. The mixing device 16 mixes the cooled intermediate liquid phase hydrocarbon stream 58 with the combined stream 54 that includes the partially cooled, compressed net gas phase stream 50 and the light ends stabilizer stream 52 to form a two-phase combined stream 62. In an exemplary embodiment, the mixing device 16 aggressively mixes or homogenizes the combined stream 54 into the cooled intermediate liquid phase hydrocarbon stream 58 such that the gas phase is broken up into a plurality of small bubbles that are well dispersed in turbulent flow with a C₅⁺ hydrocarbon-rich liquid phase so that C₃/C₄ hydrocarbons are readily extracted from the gas phase to the C₅⁺ hydrocarbon-rich liquid phase. As such, the two-phase combined stream 62 has a gas phase that is rich in H₂ and substantially depleted of C₃⁺ hydrocarbons and a liquid phase that is rich in C₃⁺ hydrocarbons.

The mixing device 16 may be a static mixer, a jet mixer, or the like. In an exemplary embodiment, the mixing device 16 is a jet mixer and the cooled intermediate liquid phase hydrocarbon stream 58 is a motive liquid at higher pressure than the combined stream 54 (e.g., the partially cooled, compressed net gas phase stream 50 and/or the light ends stabilizer stream 52) and moves rapidly through the jet mixer causing the combined stream 54 to be sucked into the jet mixer by a “Venturi effect,” such as occurs in an eductor or ejector, as a suction fluid to homogenize the combined stream 54 in the cooled intermediate liquid phase hydrocarbon stream 58. Various suitable jet mixers are commercially available. Other suitable static or jet mixers known to those skilled in the art may also be used.

In an exemplary embodiment, little or no pressure drop occurs through the mixing device 16 (e.g., jet mixer) as the cooled intermediate liquid phase hydrocarbon stream 58 and the combined stream 54 advance therethrough to form the two-phase combined stream 62. In an exemplary embodiment, the two-phase combined stream 62 has a pressure of from about 690 to about 2,460 kPa gauge.

As illustrated, in an exemplary embodiment, the apparatus 10 comprises a bypass 64 that includes a valve 66 for diverting a portion 68 of the cooled intermediate liquid phase hydrocarbon stream 58 around the mixing device 16 for incorporation into the two-phase combined stream 62. In an exemplary embodiment, the bypass is available to control the flow ratio between the cooled intermediate liquid phase hydrocarbon stream 58 (e.g., the motive liquid stream) and the combined stream 54 (e.g., the suction gas stream), per the design limits of the mixing device 16. The bypass also enables the full cooling effect from the cooled intermediate liquid phase hydrocarbon stream 58 to be applied (for example, regardless of partitioning) to reduce the temperature and form a two-phase combined stream 70. In an exemplary embodiment, the two-phase combined stream 62 has a temperature of from about 16 to about 27° C. and the two-phase combined stream 70 has a temperature of from about 4 to about 16° C. In an exemplary embodiment, it has been found that by reducing the temperature of the two-phase combined stream 62 some of the C₃⁺ hydrocarbons in the gas phase condense into the liquid phase while H₂ and the lighter end hydrocarbons, e.g., C₂⁻ hydrocarbons, remain predominantly in the gas phase so as to further enrich the liquid phase of the two-phase combined stream 70 with C₃/C₄ hydrocarbons while further depleting the gas phase of C₃⁺ hydrocarbons.

The two-phase combined stream 70 is passed along and introduced to the recontact drum 18. While the recontacting zone 17 is illustrated as only having 2 recontact drums 18 and 20, it is to be understood that the recontacting zone 17 can have more than 2 recontact drums or only a single recontact drum. The two-phase combined stream 70 is separated in the recontact drum 18 into its corresponding gas and liquid phases to form an intermediate gas phase stream 72 that comprises H₂ and C₆⁻ hydrocarbons and an intermediate liquid phase hydrocarbon stream 74 that comprises primarily C₃⁺ hydrocarbons. In an exemplary embodiment, the intermediate gas phase stream 72 and independently the intermediate liquid phase hydrocarbon stream 74 each have a temperature of from about 4 to about 16° C. and, independently, a pressure of from about 690 to about 2,460 kPa gauge.

The intermediate gas phase stream 72 is removed from the recontact drum 18 and is passed along to a compressor 76. The compressor 76 compresses the intermediate gas phase stream 72 to form a compressed intermediate gas phase stream 78. In an exemplary embodiment, the compressed intermediate gas phase stream 78 has a temperature of from about 120 to about 160° C. and, independently, a pressure of from about 1,980 to about 5,580 kPa gauge.

The compressed intermediate gas phase stream 78 is passed along and introduced to a cooler 80 to form a partially cooled, compressed intermediate gas phase stream 82. In an exemplary embodiment, the partially cooled, compressed intermediate gas phase stream 82 has a temperature of from about 30 to about 65° C. and, independently, a pressure of from about 1,950 to about 5,550 kPa gauge.

The partially cooled, compressed intermediate gas phase stream 82 is removed from the cooler 80 and is combined with the liquid phase hydrocarbon stream 36 to form an intermediate two-phase combined stream 84. Optionally, in an exemplary embodiment, the apparatus 10 comprises a mixing device 86 (as discussed above in reference to the mixing device 16) for aggressively mixing or homogenizing the partially cooled, compressed intermediate gas phase stream 82 into the liquid phase hydrocarbon stream 36 such that the partially cooled, compressed intermediate gas phase stream 82 is broken up into a plurality of small bubbles that are well dispersed in turbulent flow with a C₅⁺ hydrocarbon-rich liquid phase of the liquid phase hydrocarbon stream 36 so that C₃/C₄ hydrocarbons are readily extracted from the gas phase to the C₅⁺ hydrocarbon-rich liquid phase. As such, the intermediate two-phase combined stream 84 has a gas phase that is rich in H₂ and substantially depleted of C₃⁺ hydrocarbons and a liquid phase that is rich in C₃⁺ hydrocarbons.

The intermediate two-phase combined stream 84 is passed along and introduced to a chiller 88. The chiller cools the intermediate two-phase combined stream 84 to form a cooled intermediate two-phase combined stream 90. In an exemplary embodiment, the cooled intermediate two-phase combined stream 90 has a temperature of from about −28 to about 4° C., such as about −12 to about 0° C., and, independently, a pressure of from about 1,920 to about 5,520 kPa gauge. In an exemplary embodiment, it has been found that by reducing the temperature of two-phase combined stream 84 some of the C₃⁺ hydrocarbons in the gas phase condense into the liquid phase while H₂ and the lighter end hydrocarbons, e.g., C₂⁻ hydrocarbons, remain predominantly in the gas phase so as to further enrich the liquid phase of the cooled intermediate two-phase combined stream 90 with C₃/C₄ hydrocarbons while further depleting the gas phase of C₃⁺ hydrocarbons.

The cooled intermediate two-phase combined stream 90 is passed along and introduced to the recontact drum 20. The cooled intermediate two-phase combined stream 90 is separated in the recontact drum 20 into its corresponding gas and liquid phases to form an H₂-rich stream 92 that comprises primarily H₂ and the cooled intermediate liquid phase hydrocarbon stream 58 as discussed above. In an exemplary embodiment, the H₂-rich stream 92 and independently the cooled intermediate liquid phase hydrocarbon stream 58 each have a temperature of from about −28 to about 4° C., such as about −12 to about 0° C., and, independently, a pressure of from about 1,920 to about 5,520 kPa gauge. In an exemplary embodiment, the H₂-rich stream 92 comprises H₂ present in an amount of from about 80 to about 95 mole % with possibly some C₄⁻ hydrocarbons.

The intermediate liquid phase hydrocarbon stream 74 is removed from the recontact drum 18 and is optionally passed through a pump 94 to the stabilizer 22. Optionally, a portion of the liquid phase hydrocarbon stream 36 may be combined with the intermediate liquid phase hydrocarbon stream 74 via line 300. The stabilizer 22 separates C₃/C₄ hydrocarbons, C₅⁺ hydrocarbons, and any remaining H₂ and C₂⁻ hydrocarbons from the intermediate liquid phase hydrocarbon stream 74 to form the light ends stabilizer stream 52, a C₃/C₄ hydrocarbon-rich LPG stream 96, and a C₅⁺ hydrocarbon-rich reformate stream 98. In an exemplary embodiment, the C₃/C₄ hydrocarbon-rich LPG stream 96 comprises C₃/C₄ hydrocarbons present in an amount of about 85 to about 99.9 mole % and the C₅⁺ hydrocarbon-rich reformate stream 98 comprises C₅⁺ present in an amount of about 90 to about 99.9 mole %. As illustrated, the C₅⁺ hydrocarbon-rich reformate stream 98 is passed through a heat exchanger 100 for indirect heat exchange with the intermediate liquid phase hydrocarbon stream 74 and is removed from the apparatus 10 as a reformate product. The C₃/C₄ hydrocarbon-rich LPG stream 96 is also removed from the apparatus 10 to be used as an LPG product. The light ends stabilizer stream 52 is passed through a valve 102 to be introduced to the partially cooled, compressed net gas phase stream 50 as discussed above.

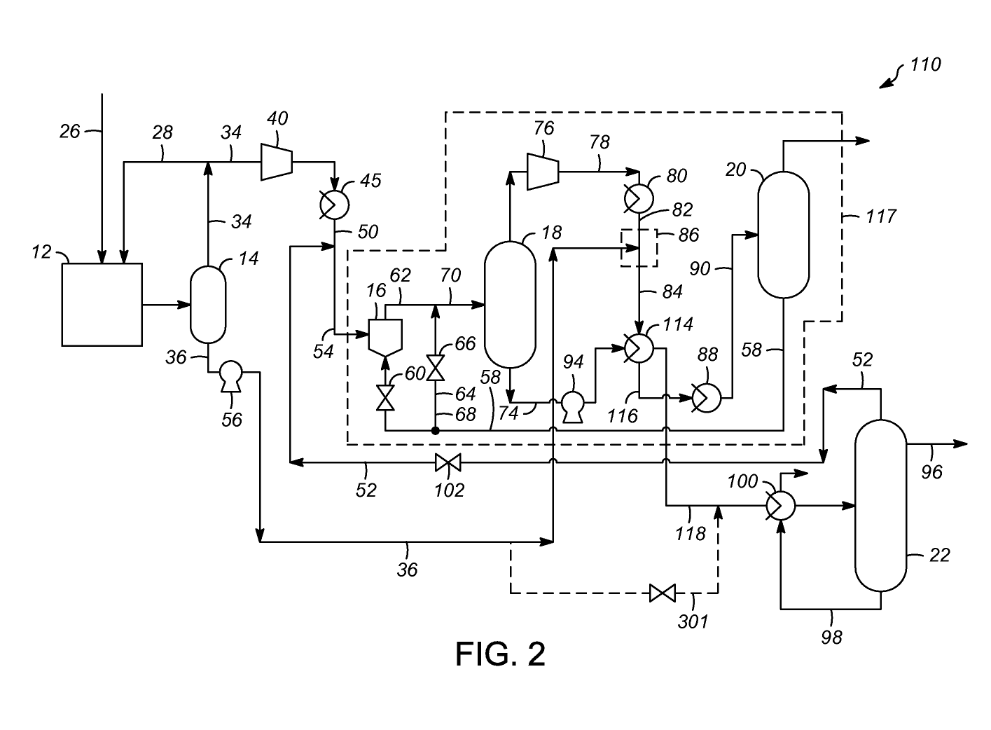

Referring to FIG. 2, an apparatus 110 for reforming of hydrocarbons in accordance with another exemplary embodiment is provided. The apparatus 110 is similarly configured to the apparatus 10 including the reforming zone 12, the separation zone 14, a recontacting zone 117 including the mixing device 16 and recontact drums 18 and 20, and the stabilizer 22 as discussed above in relation to FIG. 1 with the exception that the recontacting zone 117 further comprises an additional equipment item of a heat exchanger 114. In particular, the intermediate liquid phase hydrocarbon stream 74 exits the recontact drum 18 and is optionally passed through the pump 94 and introduced to the heat exchanger 114. In the heat exchanger 114, indirect heat exchange between the intermediate two-phase combined stream 84 and the intermediate liquid phase hydrocarbon stream 74 occurs to form a partially cooled, intermediate two-phase combined stream 116 (e.g., that includes the partially cooled, compressed intermediate gas phase stream 82 and the liquid phase hydrocarbon stream 36) and a partially heated, intermediate liquid phase hydrocarbon stream 118, respectively. In an exemplary embodiment, the partially cooled, intermediate two-phase combined stream 116 has a temperature of from about 16 to about 30° C. and a pressure of from about 1,950 to about 5,550 kPa gauge. In an exemplary embodiment, the partially heated, intermediate liquid phase hydrocarbon stream 118 has a temperature of from about 20 to about 150° C. and a pressure of from about 1,000 to about 1,500 kPa gauge. The partially cooled, intermediate two-phase combined stream 116 is passed along to the chiller 88 to form the cooled intermediate two-phase combined stream 90 as discussed above. The partially heated, intermediate liquid phase hydrocarbon stream 118 is passed through the heat exchanger 100 for indirect heat exchange with the C₅⁺ hydrocarbon-rich reformate stream 98 and is introduced to the stabilizer 22 as discussed above to form the light ends stabilizer stream 52, the C₃/C₄ hydrocarbon-rich LPG stream 96, and the C₅⁺ hydrocarbon-rich reformate stream 98. Optionally, a portion of the liquid phase hydrocarbon stream 36 may be combined with the partially heated, intermediate liquid phase hydrocarbon stream 118 upstream of the heat exchanger 100 via line 301.

Accordingly, apparatuses and methods for reforming of hydrocarbons with improved recovery of products from a reforming-zone effluent have been described. The exemplary embodiments taught herein provide a mixing device to mix a gas phase stream that is formed from a first portion of the reforming-zone effluent and that comprises H₂ and C₆⁻ hydrocarbons with a liquid phase hydrocarbon stream that is formed from a second portion of the reforming-zone effluent and that comprises C₅⁺ hydrocarbons to extract C₃/C₄ hydrocarbons from the gas phase stream into the liquid phase hydrocarbon stream and form a two-phase combined stream. The two-phase combined stream is separated for forming an H₂-rich stream that comprises primarily H₂, a C₃/C₄ hydrocarbon-rich LPG stream that comprises primarily C₃/C₄ hydrocarbons, and a C₅⁺ hydrocarbon-rich reformate stream that comprises primarily C₅⁺ hydrocarbons.

While at least one exemplary embodiment has been presented in the foregoing detailed description of the disclosure, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing an exemplary embodiment of the disclosure. It being understood that various changes may be made in the function and arrangement of elements described in an exemplary embodiment without departing from the scope of the disclosure as set forth in the appended claims.

What is claimed is:

1. An apparatus for reforming of hydrocarbons including recovery of products, the apparatus comprising:
a separation zone configured to receive and separate a reforming-zone effluent that comprises H₂, C₄⁻ hydrocarbons, and C₅⁺ hydrocarbons including aromatics to form a net gas phase stream that comprises H₂ and C₆⁻ hydrocarbons and a liquid phase hydrocarbon stream that comprises C₅⁺ hydrocarbons;
a first compressor configured to receive and compress the net gas phase stream to form a compressed net gas phase stream;
a chiller configured to receive and cool the liquid phase hydrocarbon stream to form a cooled liquid phase hydrocarbon stream; and
a first mixing device configured to receive and mix the compressed net gas phase stream and at least a portion of the cooled liquid phase hydrocarbon stream to extract C₃/C₄ hydrocarbons from the compressed net gas phase stream into the at least the portion of the cooled liquid phase hydrocarbon stream to form a first two-phase combined stream.

2. The apparatus of claim 1, wherein the first mixing device is a static mixer or a jet mixer.

3. The apparatus of claim 1, wherein the first mixing device is a jet mixer that is configured to receive the at least the portion of the cooled liquid phase hydrocarbon stream as a motive liquid and the compressed net gas phase stream as a suction fluid to homogenize the compressed net gas phase stream in the at least the portion of the cooled liquid phase hydrocarbon stream forming the first two-phase combined stream.

4. The apparatus of claim 1, further comprising a first cooler that is downstream from the first compressor and upstream from the first mixing device, wherein the first cooler is configured to receive and partially cool the compressed net gas phase stream to form a partially cooled, compressed net gas phase stream, and wherein the first mixing device is configured to receive and mix the partially cooled, compressed net gas phase stream with the at least the portion of the cooled liquid phase hydrocarbon stream to form the first two-phase combined stream.

5. The apparatus of claim 1, further comprising a first recontact drum that is configured to receive and separate the first two-phase combined stream to form an intermediate gas phase stream and a first intermediate liquid phase hydrocarbon stream.

6. The apparatus of claim 5, further comprising:
a second compressor configured to receive and compress the intermediate gas phase stream to form a compressed intermediate gas phase stream;
a second cooler configured to receive and partially cool the compressed intermediate gas phase stream to form a partially cooled, compressed intermediate gas phase stream, and wherein the chiller is configured to receive and cool the partially cooled, compressed intermediate gas phase stream with the liquid phase hydrocarbon stream to form a cooled intermediate two-phase combined stream; and
a second recontact drum in fluid communication with the chiller and the first mixing device, wherein the second recontact drum is configured to receive and separate the cooled intermediate two-phase combined stream to form an H₂-rich stream and a cooled second intermediate liquid phase hydrocarbon stream, and wherein the first mixing device is configured to receive and mix the compressed net gas phase stream and the cooled second intermediate liquid phase hydrocarbon stream to form the first two-phase combined stream.

7. The apparatus of claim 6, further comprising a second mixing device that is in fluid communication with the separation zone, the second cooler, and the chiller, wherein the second mixing device is configured to receive and mix the partially cooled, compressed intermediate gas phase stream with the liquid phase hydrocarbon stream to extract C₃/C₄ hydrocarbons from the partially cooled, compressed intermediate gas phase stream into the liquid phase hydrocarbon stream and form an intermediate two-phase combined stream, and wherein the chiller is configured to receive and cool the intermediate two-phase combined stream to form the cooled intermediate two-phase combined stream.

8. The apparatus of claim 6, further comprising a bypass that is in fluid communication with the second recontact drum and the first recontact drum, wherein the bypass is configured to divert a first portion of the cooled second intermediate liquid phase hydrocarbon stream around the first mixing device for incorporation into the first two-phase combined stream downstream from the first mixing device and upstream from the first recontact drum.

9. The apparatus of claim 6, further comprising a stabilizer in fluid communication with the first recontact drum, wherein the stabilizer is configured to receive and separate the first intermediate liquid phase hydrocarbon stream to form a C₃/C₄ hydrocarbon-rich LPG stream, a light ends stabilizer stream, and a C₅⁺ hydrocarbon-rich reformate stream.

10. The apparatus of claim 9, wherein the apparatus is configured to combine the partially cooled, compressed intermediate gas phase stream and the liquid phase hydrocarbon stream downstream from the second cooler to form an intermediate two-phase combined stream, wherein the apparatus further comprises a heat exchanger in fluid communication with the first recontact drum, the second cooler, the chiller, and the stabilizer, and wherein the heat exchanger is configured for indirect heat exchange between the intermediate two-phase combined stream and the first intermediate liquid phase hydrocarbon stream to form a partially cooled, intermediate two-phase combined stream and a partially heated, first intermediate liquid phase hydrocarbon stream, respectively, and wherein the chiller is configured to receive and cool the partially cooled, intermediate two-phase combined stream to form the cooled intermediate two-phase combined stream and the stabilizer is configured to receive the partially heated, first intermediate liquid phase hydrocarbon stream to form the C₃/C₄ hydrocarbon-rich LPG stream and the C₅⁺ hydrocarbon-rich reformate stream.

11. A method for reforming of hydrocarbons including recovery of products, the method comprising the steps of:
separating a reforming-zone effluent that comprises H₂, C₄⁻ hydrocarbons, and C₅⁺ hydrocarbons including aromatics to form a net gas phase stream that comprises H₂ and C₆⁻hydrocarbons and a liquid phase hydrocarbon stream that comprises C₅⁺ hydrocarbons;
compressing the net gas phase stream to form a compressed net gas phase stream;
cooling the liquid phase hydrocarbon stream to form a cooled liquid phase hydrocarbon stream; and
mixing the compressed net gas phase stream with at least a portion of the cooled liquid phase hydrocarbon stream in a mixing device to extract C₃/C₄ hydrocarbons from the compressed net gas phase stream into the at least the portion of the cooled liquid phase hydrocarbon stream to form a first two-phase combined stream.

12. The method of claim 11, wherein the step of compressing comprises forming the compressed net gas phase stream having a pressure of from about 720 to about 2,490 kPa gauge.

13. The method of claim 11, wherein the step of compressing comprises forming the compressed net gas phase stream having a temperature of from about 120 to about 150° C.

14. The method of claim 11, wherein the step of cooling comprises forming the cooled liquid phase hydrocarbon stream having a temperature of from about −28 to about 4° C.

15. The method of claim 11, wherein the step of mixing comprises forming the first two-phase combined stream having a temperature of from about 16 to about 27° C.

16. The method of claim 11, wherein the step of mixing comprises forming the first two-phase combined stream having a pressure of from about 690 to about 2,460 kPa gauge.

17. The method of claim 11, further comprising the step of separating the first two-phase combined stream to form an intermediate gas phase stream that comprises primarily H₂ and some C₆⁻ hydrocarbons and an intermediate liquid phase hydrocarbon stream that comprises primarily C₃⁺ hydrocarbons.

18. The method of claim 17, further comprising the step of bypassing a first portion of the at least the portion of the cooled liquid phase hydrocarbon stream about the mixing device for incorporation into the first two-phase combined stream to reduce a temperature of the first two-phase combined stream prior to the step of separating the first two-phase combined stream.

19. The method of claim 18, wherein the step of bypassing comprises incorporating the portion of the at least the portion of the cooled liquid phase hydrocarbon stream into the first two-phase combined stream to reduce the temperature to about 4 to about 16° C. for separating the first two-phase combined stream into the intermediate gas phase stream and the intermediate liquid phase hydrocarbon stream.

20. A method for reforming of hydrocarbons including recovery of products, the method comprising the steps of:
mixing a gas phase stream that comprises H₂ and C₆⁻ hydrocarbons with a liquid phase hydrocarbon stream that comprises C₅⁺ hydrocarbons in a jet mixer to extract C₃/C₄ hydrocarbons from the gas phase stream into the liquid phase hydrocarbon stream and to form a two-phase combined stream; and
separating the two-phase combined stream for forming an H₂-rich stream that comprises primarily H₂, a C₃/C₄ hydrocarbon-rich LPG stream that comprises primarily C₃/C₄ hydrocarbons, and a C₅⁺ hydrocarbon-rich reformate stream that comprises primarily C₅⁺ hydrocarbons.
